(19) United States
(12) Patent Application Publication
(10) Pub. No.: US 2004/0172049 A1
(43) Pub. Date: Sep. 2, 2004

(54) Vascular anastomosis device

(76) Inventor: Hoon-Bum Lee, Kangnam-gu (KR)

Correspondence Address: Mavis S Gallenson, Ladas & Parry, Suite 2100, 5670 Wilshire Boulevard, Los Angeles, CA, 90036-5679, US

(21) Appl. No.: 10/479,287

(22) PCT Filed: Jun. 7, 2002
(86) PCT No.: PCT/KR02/01078

(30) Foreign Application Priority Data
Jun. 7, 2001 (KR) 2001-31783

(51) Int. Cl. A61F 2/06

(52) U.S. Cl. 606/153; 604/8; 623/1.36

(57) ABSTRACT

The present invention provides a novel vascular anastomosis device capable of anastomosing readily the side (lateral portion) of a vessel to the side of another vessel. By employing the vascular anastomotic device of the present invention, it is possible to anastomose two vessels in the side to-side type which is generally very difficult in the art associated with anastomosis, by simple procedure without using a suture, whereby the operative time decreases remarkably. Furthermore, since the operative site needs not to be opened fully, a patient's pain can be released.

### TECHNICAL FIELD

The present invention relates to a vascular anastomosis device, more particularly, the vascular anastomosis device capable of anastomosing readily the side (lateral portion) of a vessel to the side of another vessel.

### BACKGROUND ART

The lack of exercise and occidentalization of eating habits are factors of increasing the incidence rate of vascular diseases from year to year. In particular, most of vascular diseases are cardiac infarction and angina pectoris caused by the ischemia that a blood vessels is clogged feeding nutriment and oxygen to the heart.

In order to treat the clogged vessel or almost clogged vessel, the pharmaco-therapy using muscle relaxants or anti-calcium drugs for extension of vessel muscle and the operative-therapy have been practiced. The pharmaco-therapy is useful for treatment at the initial symptom but is not so when the clogged step was progressed to the rather extent; therefore, the pharmaco-therapy is not substantial treatment and the operative-therapy has been generally practiced being the direct treatment of clogged vessel.

The operative-therapy is categorized into an internal operation and a surgical operation. The internal operation is to insert a stent into the clogged portion (atresia portion) through a blood vessel such as the femoral vessel and then expand the stent. The method of using an expansible stent and apparatuses therefor are disclosed in U.S. Pat. No. 3,416,531. However, when a certain time elapses after the internal operation according to such method, lesion tissues protrude between nets of the stent and new granular tissues grow on the inner surface of stent, whereby the atresia phenomenon reoccurs.

Accordingly, as a further essential solution to this problem, the surgical operation has been carried out opening the heart and connecting the atresia vessel by a bypass with a vessel extracted from other organ of a patient (mainly, leg vein). At this surgical operation, one end of the vein is connected to IMA (Internal Mammary Artery: the artery feeding nutriment and oxygen to the organ and muscle of the chest and abdomen) by the end-to-end or end-to-side way, and the other end is connected to the vessel beyond the atresia portion by the end-to-side or side-to-side way.

Meanwhile, a blood vessel comprises the intima, the media and the adventitia, and at anastomosis of two vessels, the intima should be connected to the intima with these vessels everted. For anastomosis of severed vessels, and reconstruction by the skin flap transplantation, as well as treatment of cardiac diseases like the above, the specialist in the microsurgery secures a magnified operative visual-field with a microscope or a powerful magnifier and then sutures vessels or tissues one by one with stitching fibers. The suture can be carried out only by the microsurgical specialist and thus is very time-consuming and heavy work. In particular, it is very difficult to suture one by one vessels being in the portion like the heart which pulsates periodically. Accordingly, it is necessary to stop the pulsation of heart for a long time (at least more than 3 hours) by causing the heart attack intentionally at the operation.

Therefore, various methods have been developed to perform anastomosis with mechanic devices in a short time without resorting to suturing. Theses examples of mechanic devices include ones disclosed in U.S. Pat. No. 3,774,615, U.S. Pat. No. 4,214,586, U.S. Pat. No. 4,366,819, U.S. Pat. No. 4,523,592, U.S. Pat. No. 4,917,087, U.S. Pat. No. 5,752,966, U.S. Pat. No. 5,817,113, U.S. Pat. No. 6,206,913, etc. However, all of these patents are directed to devices for the end-to-end and end-to-side anastomoses and cannot be used to the side-to-side anastomosis.

The side-to-side anastomosis is very difficult compared with the other anastomoses; however, so far, devices for the side-to-side anastomosis have not been invented.

### SUMMARY OF INVENTION

The objects of the present invention are to solve the problems described above for once and all.

An object of the present invention is to provide a device capable of anastomosing the side of a vessel to the side of other vessel readily without use of a suture (stitching fiber).

Another object of the present invention is to provide a device capable of conducting the side-to-side anastomosis without opening the operative site fully.

In order to accomplish these objects, a vascular anastomotic device of the present invention for side-to-side anastomosis comprises, a pair of assembly members being able to join and disjoin along its axial plane, the joined assembly members generally being of a tubular shape of which the bottom is closed, wherein each assembly member comprises upper and lower units;

a protruding member installed at the upper portion of each assembly member, the protruding member having a plurality of hooking means which are positioned downwardly along the circumferential surface of the bottom of the protruding member;

a connecting member installed inside each assembly member between the upper and lower units, the connecting member connecting the upper and lower units when these units are separated by the pulling force applied to the device; and

semicircular openings formed at both sides of each assembly member, the semicircular openings of an assembly member making two circular openings together with the semicircular openings of the corresponding assembly member when a pair of assembly members join, wherein the two circular openings provide a passage penetrating the assembly members laterally.

When a pair of assembly members joins to each other, the vascular anastomosis device of the present invention forms generally a tubular shape of which the bottom is closed, and the two circular openings provide the passage penetrating the device laterally. In the state of joining, when the device is pulled in the axial direction, it extends to the length defined by the connecting member. For side-to-side anastomosis, while a vessel (A) is inserted into the passage provided by the two openings, other vessel (B) is engaged to the hooking means of the protruding member. In order to engage the vessel (B) to the hooking means, the vessel (B) is incised longitudinally to make an incision and then the protruding member is inserted through the incision. Accordingly, when the protruding member is of an oval shape in its cross-section, the procedure of inserting the protruding member through the incision become easier. The more detailed procedure will be illustrated later referring to embodiments on the drawings.

On a vertical surface on which the two assembly members come into contact with each other, are installed joint means. The joint means can have a diversity of configurations to join the two assembly members to each other; however, in consideration of the feature of the fine vascular anastomosis, the joint means is preferably configured to be able to be readily engaged by one-time applying force. Accordingly, one preferable embodiment of the joint means is a pair of male-female joints, the male joint being of an anchor shape, and the female joint being of a recess into which the male joint can be inserted. If the assembly members are made of materials having the plasticity, the anchor-like joint can be inserted into the vertical surface of corresponding assembly member, although said recess is not formed thereon. Preferably, the two assembly members are connected by a hinge at the bottom of them, so that the upper of the two assembly members are spread in the state of disjoint, whereby interlock of the two assembly members can be accomplished by folding them. For engagement of a vessel (A), the vessel (A) is positioned on the spread upper portion of the device, more particularly, on the semicircular openings thereof, and then the spread upper portion is folded to be able to accomplish the engagement easily.

The connecting member acts as connecting the upper and lower units which are separated by a certain length, as the pulling force is applied to the device. In a preferable embodiment, the connecting member comprises a main body of a certain length, both ends of the main body being smaller than the width of a guiding road but larger than the width of an entry of the guiding road, wherein the guiding road is formed inside along a wall of the upper and lower units, respectively, and the main body extends through entries of the upper and lower units to these guiding roads. Therefore, when a pulling force is not applied to the device, the upper unit comes in contact with the lower unit, and the connecting member is positioned on the guiding roads of these units. Meanwhile, when a pulling force is applied, the upper and lower units are separated, and the connecting member appears in the space between these units separated. The maximum length of separation is defined by the length of the main body of connecting member and, in the maximum separation state, both ends of the connection member are hung on the entries of guiding roads. The width of the entry of guiding road is smaller than that of the end of connecting member, so that the connecting member is not detached from the upper and lower units, although a big pulling force is applied thereto.

In desirable embodiments, the following configurations can be used to render engagement of vessels to the hooking means stronger.

In an exemplary configuration, a ring-mounting member protrudes from the side of the assembly member in the position between the protruding member and the semicircular opening, the ring-mounting member being curved upwardly. In the anastomotic procedure, a resilient silicone ring is mounted on the ring-mounting member. The silicone ring which has been mounted on the member moves forward, in the time of engagement of vessel (B), along the curved surface of member to make the engagement of vessel (B) to the hooking means of the protruding member stronger.

In another exemplary configuration, a flat surface (“guiding surface”) is formed between the protrusion member and semicircular member, and a sliding member is mounted moving on the guiding surface upwardly and downwardly. The sliding member, which is of a ring shape and surrounds the outer surface of the interlocked assembly members, comprises a pair of joinable units, each unit having joints on both ends thereof, or each unit having a hinge on one end thereof to connect itself with the corresponding unit and having a joint on the other end. In the anastomotic procedure, the sliding member mounted on the guiding road serves as making the engagement of vessel (B) to the hooking means stronger by moving forward when the vessel (B) is engaged to the hooking means. Herein, the sliding member has preferably recesses on its proximal surface for insertion of the hooking means of the protruding member. However, in the case that the sliding member is made of soft materials, the hooking means can be inserted into the proximal surface of the sliding member having no recesses.

Since the vascular anastomotic device of the present invention will be permanently installed in a human body after anastomosis, it should be of biocompatible so that nontoxic metals such as titanium or silicone, etc. can be used as materials. Also, biodegradable materials which have been known in the art can be used.

As shown below, the description refers to the drawing in order to describe the present invention more in detail, thereby, the scope of the invention is however not to be interpreted as a limitation of the invention.

### DETAILED DESCRIPTION OF PREFERRED EMBODIMENTS

FIG. 1A shows a vascular anastomotic device according to the embodiment of the present invention. A vascular anastomotic device 100 comprises a pair of assembly members 200 being able to join and disjoin. Each assembly member 200 comprises a protruding member 210 having a plurality of hooks 300; a ring-mounting member 220 for a silicone ring 400 to be mounted in anastomotic procedure; and upper and lower units 230 being able to separate by a pulling force applied upwardly and downwardly. Also, circular openings 500 are formed in the position that the assembly members meet. The assembly members 300 interlocked form the horizontal cross-section of an oval shape to be readily inserted through an incision for anastomosis into a vessel.

The hooks 300 are downward installed along a circumferential surface 212 of the bottom of the protruding member 210. Referring to FIGS. 4D and 4F, for easy engagement of vessels (A), (B), the heads of hooks 300 are preferably bending inwardly.

The ring-mounting member 220 are bending to form a curved surface upwardly so that the silicone ring 400 mounted thereon in anastomotic procedure can move toward the protruding member 210 readily.

Turning to FIG. 1B, this figure shows a vertical cross-section along a-a line of FIG. 1A. In FIG. 1B, the configuration can be illustrated that the upper and lower units 232, 234 are connected in an extensible form. A guiding road 236 of a certain length is formed in the upper and lower units 232, 234, and the connecting member 600 of a certain length extends through the guiding road 236 of these units 232, 234. The connecting member 600 comprises a body 610 having a length corresponding to the sum of lengths of the two guiding roads 236, and both ends 620 having a larger dimension than the width of the body 610.

FIG. 1C shows the configuration in which a pair of assembly members are entirely detached. The assembly members consist of a left assembly member 202 and a right assembly member 204, which are symmetrical to each other and have joints 240 on vertical surfaces meeting with each other. The preferred configuration of the joints 240 consists of a male joint 242 of an anchor shape and a female joint 244 of a recess corresponding to the male joint 242. When the male joint 242 is installed on the left assembly member 202, the female joint 244 is installed on the right assembly member 204.

FIG. 2A shows a vertical cross-section of a vascular anastomosis device 110 according to another embodiment of the present invention. The anastomosis device 110 does not have a silicon ring-mounting member. That is, the engagement of the anastomosis device 110 to a vessel (B) is accomplished only by hooks 300 and a separate silicone ring is not used for making such engagement stronger.

FIG. 2B shows a vertical cross-section of a vascular anastomosis device 120 according to another embodiment of the present invention. In the anastomosis device 120, a flat surface (guiding surface: 122) is formed on the outer surface between a protruding member 210 and a circular opening 500, and a sliding member 450 is mounted on the guiding surface 122. The sliding member 450 can move upward and downward along the guiding surface 122. An exemplary configuration of the sliding member 450 will be illustrated in below together with the device 120. The sliding member 450 comprises a pair of joinable units 451, 452 and has a cross-sectionally oval shape like that of an assembly unit, the joinable units 451, 452 being connected by a hinge 453 in one side of ends thereof, so that these units 451, 452 can pivot on the hinge 453. Furthermore, the sliding member 450 has on its proximal plane a plurality of recesses 456 into which hooks 300 of the protruding member 210 can insert. In another embodiment, for the purpose of further facilitating an endoscopic operative procedure employing the anastomotic device 120, a holding means 124 being of a protruding form (as in the figure) or caving-in form is formed on the outer surface of the device 120, which can be readily held by a separate device (not shown).

FIG. 2C shows a vertical cross-section of a vascular anastomotic device 130, being a modification of the anastomotic device 120 in FIG. 2B, which is connected by a hinge 203 installed at the bottom of a pair of assembly units 201, 202. Accordingly, in the non-interlocked state, the upper portion of the device 130 is spread so that the spread anastomotic device 130 is placed on a vessel (13) and is then interlocked by folding them. Herein, the vessel (B) must be positioned in an opening 500.

FIG. 3A shows the transform of a vascular anastomotic device 100 when a pulling force is applied to the upward and downward direction. As the upward-downward pulling force is applied to the anastomotic device 100, upper and lower units 232, 234 constituting the body of device are separated and a connecting member appears on the space between these units separated.

FIG. 3B, being a cross-sectional view along the line b-b of FIG. 3A, shows the principle that the upper and lower units 232, 234 are separated and these units are connected by the connecting member 600. More specifically, as the pulling force is applied, the upper and lower units 232, 234 are separated, and the connecting member 600 which has been on a guiding road 236 of the upper and lower units 232, 234 appears on the spaced portion. Herein, what appears outside is a body 610 of the connecting member 600, and the maximum separated length is defined by the length of the body 610. In the entirely separated state, both ends 620 of the connecting member 600 reach entries 238 of the guiding roads 236, the ends 620 having the larger dimension than the entry 238, whereby the body 610 of the connecting member 600 does not come entirely off the guiding road 236.

The body 610 of the connecting member 600, as shown in FIG. 3A, is of a cylindrical shape being identical with the lateral portion of the device 100, and the bottom of the device 100 is closed, so that the expansion of a vessel (A: not shown) which has been inserted into a circular opening 500, caused by the blood pressure, occurs only on the open upper portion of the anastomotic device 100.

FIGS. 4A to 4I show a series of procedures of anastomosing the lateral portion of a vessel (A) to the lateral portion of a vessel (B) employing the vascular anastomotic device 100 of FIG. 1A.

In the step of FIG. 4A, left and right assembly units constituting the vascular anastomotic device 100 are interlocked on the vessel (A). For the convenience of explanation, the anastomotic device 100 is joined in the reverse direction. Herein, the diameter of an circular opening (not shown) of the anastomotic device 100 is at least identical with or preferably smaller than the outer diameter of the vessel (A) so that the pressure of blood flowing through the vessel (A) (“blood pressure”) works at the center of the anastomotic device 100, thereby a part of the vessel (A) protruding from an open upper portion 214 of the device 100 as shown in FIG. 1A. It should be noted that a blood vessel can generally expand as many as twice and then recover without any strain. A resilient silicone ring 400 is mounted on the ring-mounting member 220. This procedure may be performed in the step of FIG. 4D wherein the expanded vessel is engaged to hooks.

In the step of FIG. 4B, when the blood flow in the vessel (A) is of the arrow direction, a blocking device 700 is installed at the position beyond the device 100. Accordingly, the high pressure works on the vessel (A) around the device 100 so that the vessel (A) expands, whereby the vessel part (Aa) of the open upper portion 214 expands further.

When a vessel part (Aa) has expands to the appropriate extent, in the step of FIG. 4C, in order to block the further introduction of blood, another blocking device 700 is also installed at the position before the device 100, then, the vessel part having protruded is incised.

In the step of FIG. 4D, the incised vessel part (Aa) is everted to be engaged to hooks 300 of a protruding member 210, thereby the intima of the vessel par (Aa) being exposed. The exposition of intima is necessary for vascular anastomosis by intima-to-intima joint as mentioned earlier. To mount a silicon ring 400 may be performed after completing engagement of the vessel (A).

In the step of FIG. 4E, when the blood flow in the vessel (B) is of the arrow direction, a blocking device 700 is installed on a vessel (B) and the vessel part beyond the blocking device 700 is incised in the longitudinal direction. The length of an incision 800 of the vessel (B) is preferably smaller than the outer diameter of the protruding member 210. Since a vessel is generally elastic, the protruding member 210 can be inserted through the smaller incision 800 into the vessel (B) so that the possibility of bleeding decreases due to a small length of the incision 800.

In the step of FIG. 4F, the protruding member 210 of the anastomotic device 100 is inserted through the incision 800 into the vessel (B). As a result of insertion, the intima of vessel (A) comes into contact with the intima of vessel (B).

In the step of FIG. 4G, as the protruding member 210 is inserted through the incision 800, the anastomotic device 100 is pulled upward to engage the vessel (B) to the hooks 300 and herein the elastic vessel (B) is rather extended by the pulling force.

In the step of FIG. 4H, the silicone ring 400, which has mounted on the ring-mounting member 220, moves forward the vessel (B), so that the silicone ring 400 makes engagement of the vessels (A), (B) stronger by covering the vessel (B) which has been engaged to the hooks 300 of the protruding member 210. Finally, the blocking devices 700 are removed from the vessels (A), (B).

In the step of FIG. 4I, as the blood flows through the vessels (A), (B) which all the blocking devices have been removed from, the blood pressure works again on the vascular anastomotic device 100, so that upper and lower units 232, 234 are separated and a connecting member 600 appears which has been inside the these units 232, 234. Accordingly, the circular opening (not shown) of the anastomotic device increases to the extent corresponding to the diameter of the vessel (A) The present invention being thus described, it will be obvious that the same may be varied in many ways. Such variations are not to be regarded as a departure from the spirit and scope of the invention and all such modifications would be obvious to one skilled in the art.

### BRIEF DESCRIPTION OF THE DRAWINGS

FIGS. 1A to 1C are perspective and vertical cross-sectional views of a vascular anastomotic device according to an embodiment of the present invention.

FIGS. 2A to 2C are vertical cross-sectional views of a vascular anastomotic device according to another embodiment of the present invention.

FIGS. 3A and 3B are perspective and vertical cross-sectional views of the vascular anastomotic device of FIG. 1A to which a pulling force has been applied upward and downward.

FIGS. 4A to 4I are views of a series of procedures of anastomosing two vessels in the side-to-side type by employing the vascular anastomotic device of FIG. 1A.

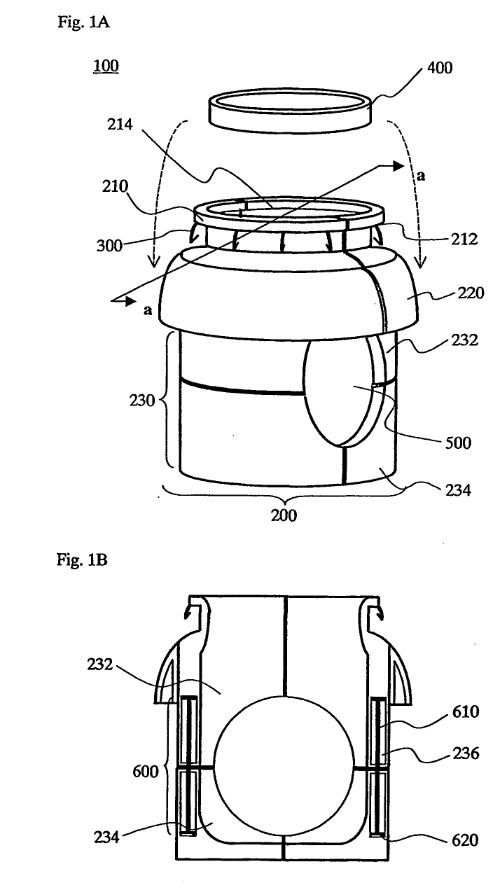

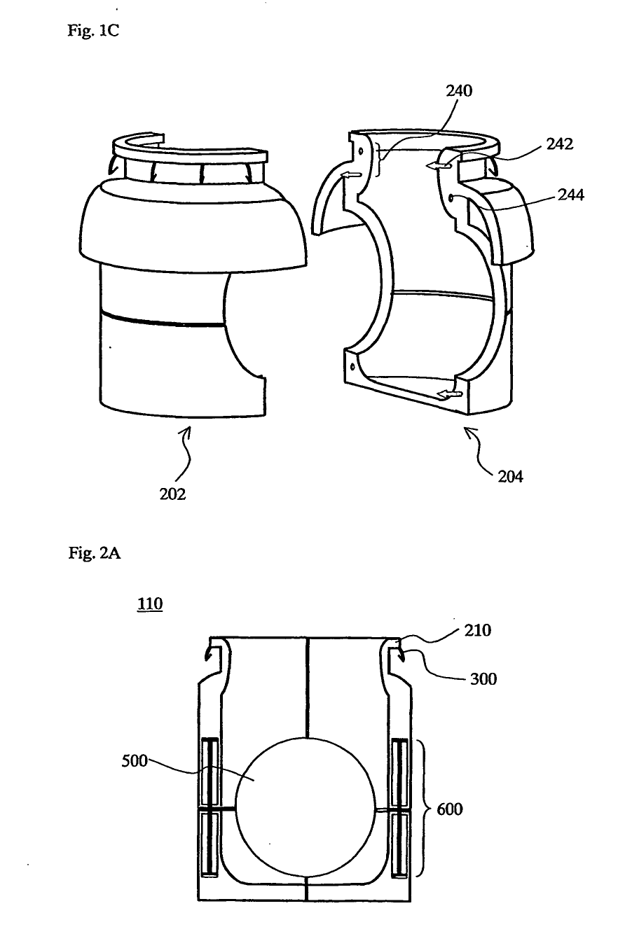

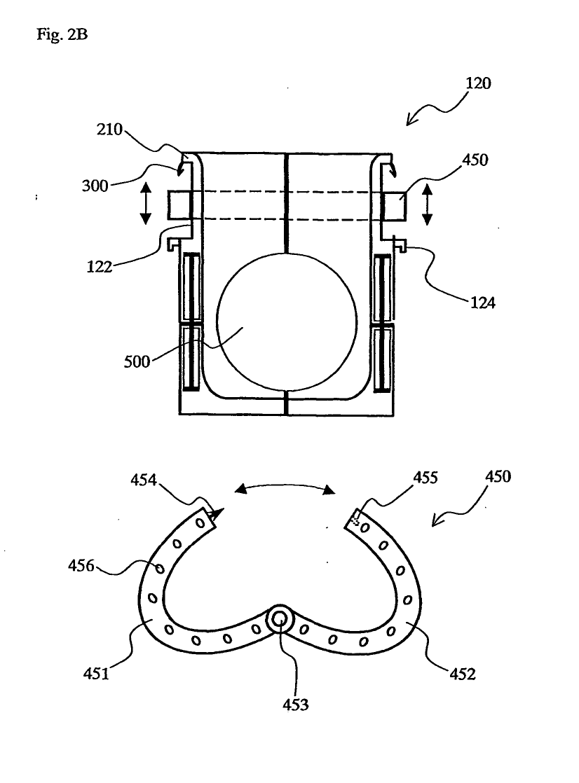

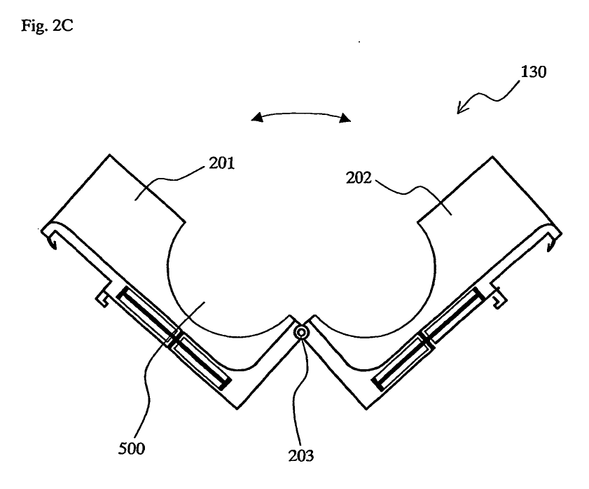

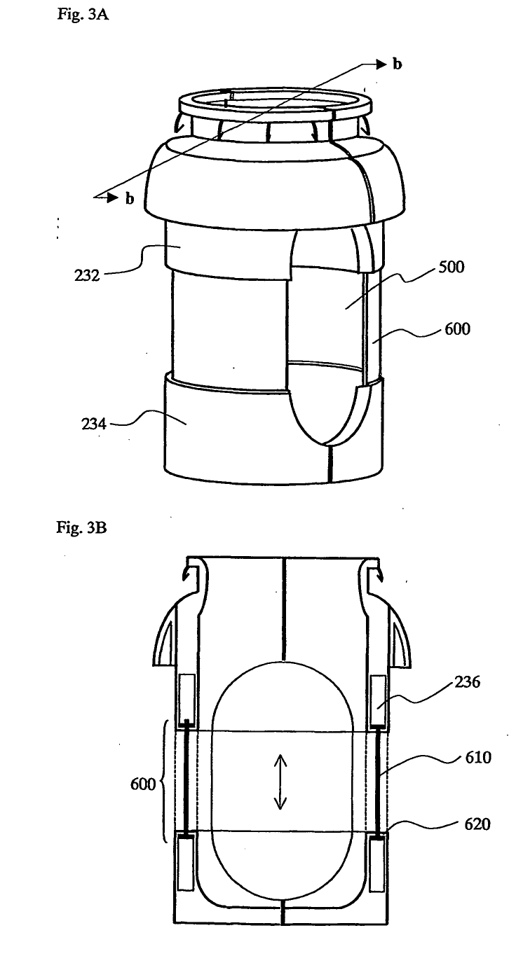

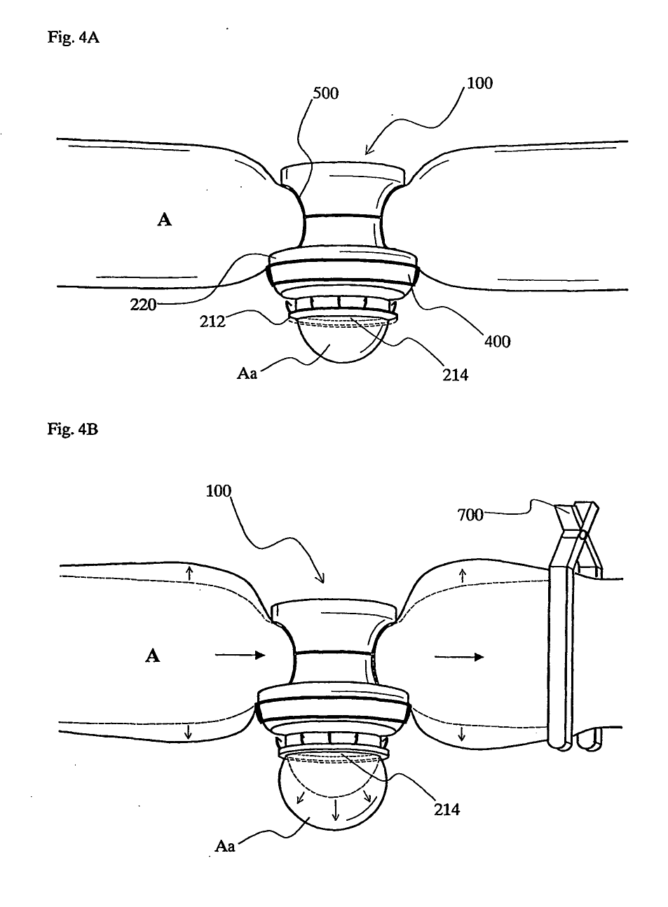

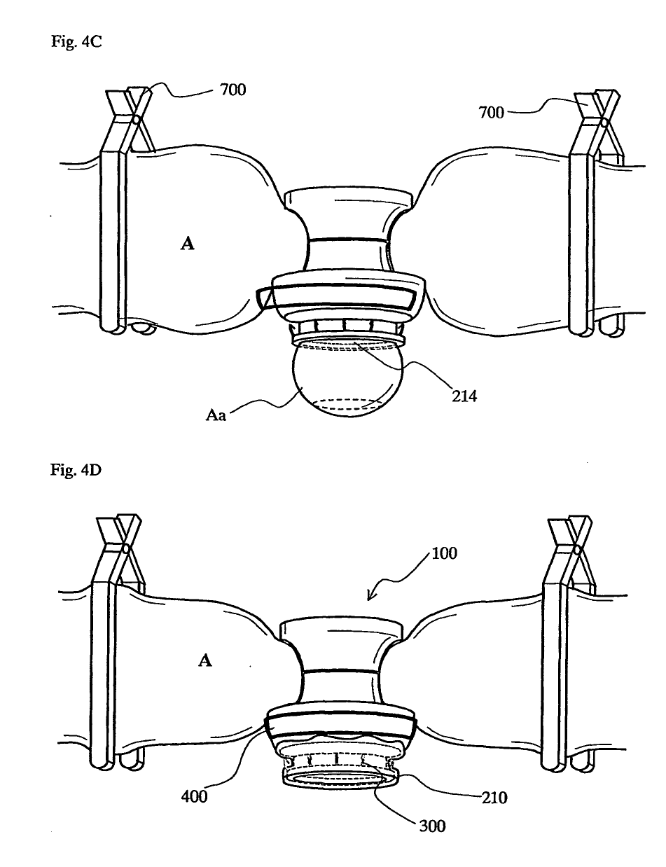

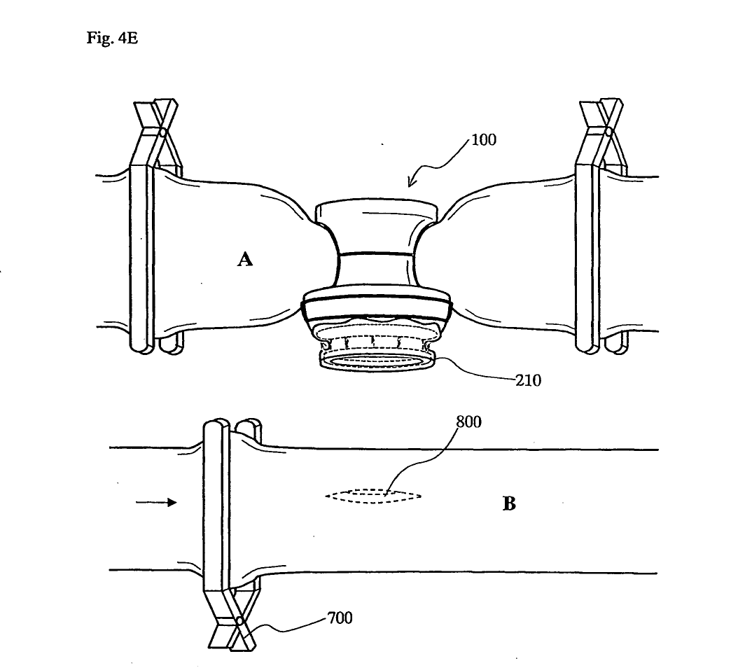

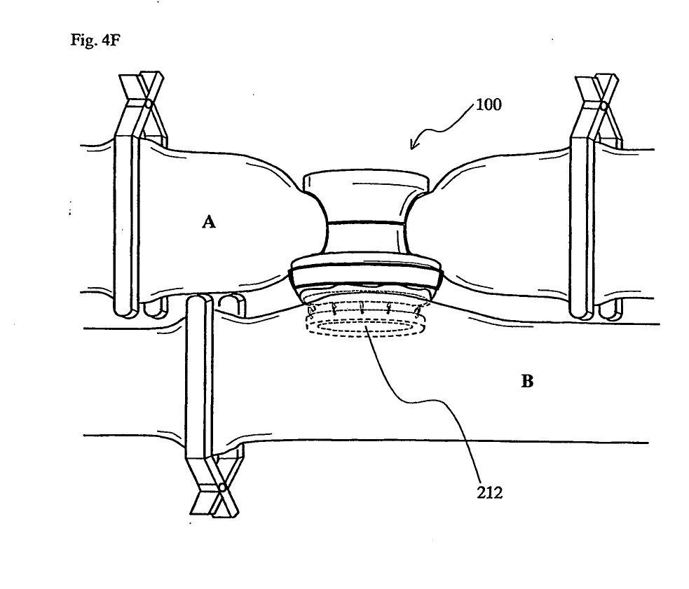

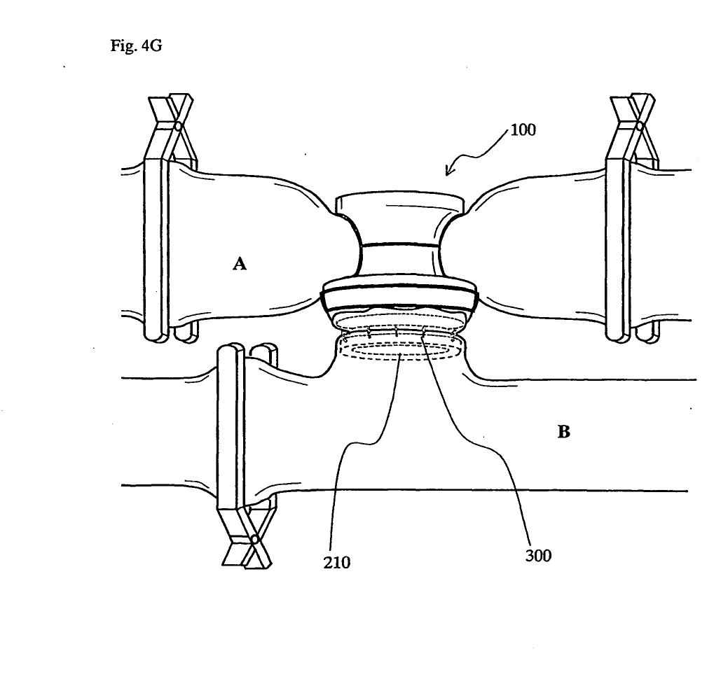

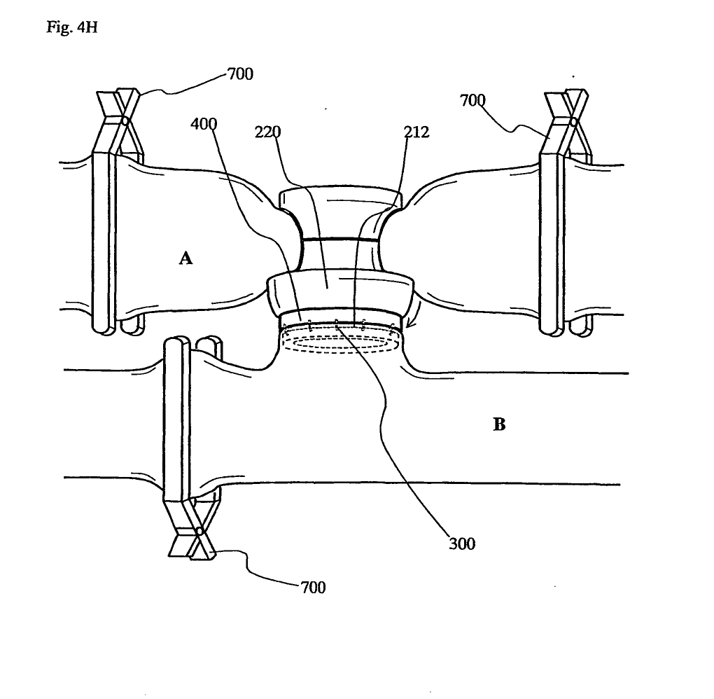

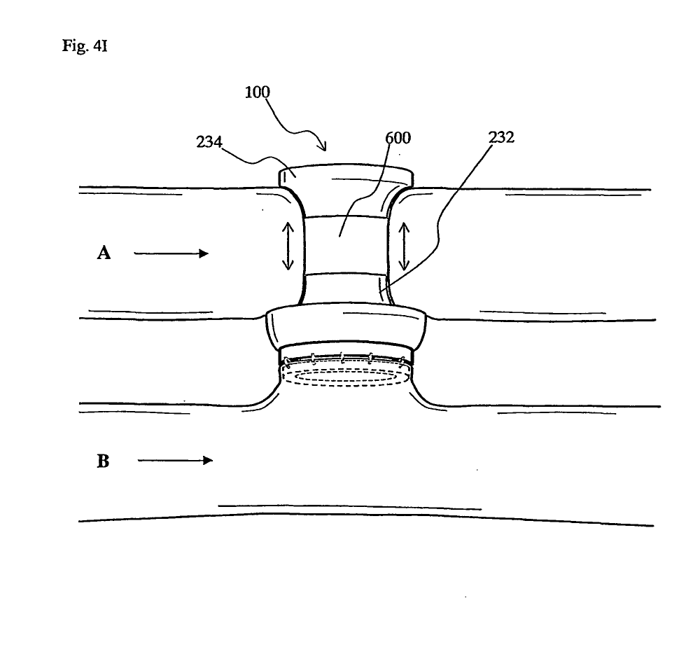

### DESIGNATION OF THE REFERENCE NUMBERS

100: vascular anastomotic device

200: assembly member

300: hook

400: silicone ring

500: opening

600: connecting member

700: blocking device

800: incision

### INDUSTRLIAL APPLICABILITY

By employing the vascular anastomotic device of the present invention, it is possible to anastomose two vessels in the side-to-side type, which is generally very difficult in the art associated with anastomosis, by a simple procedure without using a suture, whereby the operative time decreases remarkably. Furthermore, since the operative site needs not to be opened fully, a patient's pain can be released. In particular, the side-to-side anastomotic device is the novel one in the relevant art.

What is claimed is:

1. A vascular anastomotic device for side-to-side anastomosis comprising,
a pair of assembly members being able to join and disjoin along its axial plane, the joined assembly members generally being of a tubular shape of which the bottom is closed, wherein each assembly member comprises upper and lower units;
a protruding member installed at the upper portion of each assembly member, the protruding member having a plurality of hooking means which are positioned downwardly along the circumferential surface of the bottom of the protruding member;
a connecting member installed inside each assembly member between the upper and lower units, the connecting member connecting the upper and lower units when these units are separated by the pulling force applied to the device; and
semicircular openings formed at both sides of each assembly member, the semicircular openings of an assembly member making two circular openings together with the semicircular openings of the corresponding assembly member when a pair of assembly members join, wherein the two circular openings provide a passage penetrating the assembly members laterally.

2. The vascular anastomotic device according to claim 1, wherein joint means are installed on a vertical surface on which the two assembly members come into contact with each other, and the joint means comprises a pair of male-female joints, the male joint being of an anchor shape, and the female joint being of a recess into which the male joint can be inserted.

3. The vascular anastomotic device according to claim 1, wherein a ring-mounting member being curved upwardly protrudes from the side of the assembly member in the position between the protruding member and the semicircular opening, and, in the anastomotic procedure, a resilient silicone ring is mounted on the ring-mounting member.

4. The vascular anastomotic device according to claim 1, wherein a flat surface (“guiding surface”) is formed between the protrusion member and semicircular member, and a sliding member moving on the guiding surface upwardly and downwardly is mounted in the anastomotic procedure, in which the sliding member, which is of a ring shape and surrounds the outer surface of the interlocked assembly members, comprises a pair of joinable units, each unit having joints on both ends thereof, or each unit having a hinge on one end thereof to connect itself with the corresponding unit and having a joint on the other end.

5. The vascular anastomotic device according to claim 4, wherein the sliding member has recesses on its proximal surface for insertion of the hooking means of the protruding member in the anastomotic procedure, or the sliding member is made of soft materials, the hooking means can be inserted into the proximal surface of the sliding member having no recesses.

6. The vascular anastomotic device according to claim 1, wherein the connecting member comprises a main body of a certain length, both ends of the main body being smaller than the width of a guiding road but larger than the width of an entry of the guiding road, in which the guiding road is formed inside along a wall of the upper and lower units, respectively, and the main body extends through entries of the upper and lower units to these guiding roads.

7. The vascular anastomotic device according to one of claims 1 to 6, wherein the two assembly members are connected by a hinge at the bottom of them, so that the upper of the two assembly members are spread in the state of disjoint, whereby interlock of the two assembly members can be accomplished by folding them.
